(19) United States
(12) Patent Application Publication
(10) Pub. No.: US 2021/0361853 A1
(43) Pub. Date: Nov. 25, 2021

(54) APPARATUSES AND METHODS FOR REMOVING FLUID FROM A WOUND UTILIZING CONTROLLED AIRFLOW

(71) Applicant: KCI Licensing, Inc., San Antonio, TX (US)

(72) Inventor: Christopher Brian LOCKE, Bournemouth (GB)

(21) Appl. No.: 16/635,638

(22) PCT Filed: Jul. 25, 2018
(86) PCT No.: PCT/US2018/043603

§ 371 Date: Jan. 31, 2020

Related U.S. Application Data
(60) Provisional application No. 62/546,866, filed on Aug. 17, 2017.

(51) Int. Cl. A61M 1/00

(52) U.S. Cl. CPC A61M 1/96; A61M 1/98; A61M 2205/3331; A61M 2205/7518; A61M 2205/7527; A61M 1/91

(57) ABSTRACT

Systems, apparatuses, and methods for providing negative pressure to a tissue site are closed. Illustrative embodiments may include a system comprising a dressing having tissue interface in fluid communication with the tissue site. Such system may also comprise a canister having a fluid inlet fluidly coupled to the canister and an ambient inlet fluidly coupled to ambient air outside the collection chamber. Such system may further comprise a first outlet fluidly coupled to the canister and adapted to receive negative pressure from a source of negative pressure, and a second outlet. Such system also may comprise a fluid conductor fluidly coupled between the second outlet and the tissue interface, wherein the fluid conductor may be adapted to deliver ambient air to the tissue site. In some embodiments, such system may also comprise a regulator fluidly coupled between the second outlet and the ambient inlet, wherein the regulator is adapted to provide ambient air through the fluid conductor to the tissue site in a controlled fashion.

### RELATED APPLICATION

This application claims the benefit, under 35 USC 119(e), of the filing of U.S. Provisional Patent Application No. 62/546,866, entitled “Apparatuses and Methods for Removing Fluid from a Wound Utilizing Controlled Airflow,” filed Aug. 17, 2017, which is incorporated herein by reference for all purposes.

### TECHNICAL FIELD

The invention set forth in the appended claims relates generally to tissue treatment systems and more particularly, but without limitation, to apparatuses and methods for providing negative-pressure therapy with instillation of topical treatment solutions.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound can be washed out with a stream of liquid solution, or a cavity can be washed out using a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and instillation therapy are widely known, improvements to therapy systems, components, and processes may benefit healthcare providers and patients.

### BRIEF SUMMARY

New and useful systems, apparatuses, and methods for instilling fluid to a tissue site in a negative-pressure therapy environment are set forth in the appended claims. Illustrative embodiments are also provided to enable a person skilled in the art to make and use the claimed subject matter. Some embodiments are illustrative of an apparatus or system for delivering negative-pressure and therapeutic solution of fluids to a tissue site, which can be used in conjunction with venting ambient air to the tissue site in a controlled fashion. For example, an apparatus may include a fluid conductor fluidly coupling a tissue site to a regulator such as, for example, a filter or valve, fluidly coupled to a source of ambient air to provide airflow through the fluid conductor to the tissue site.

In some embodiments, for example, a system for providing negative pressure to a tissue site may comprise a dressing having tissue interface in fluid communication with the tissue site and a cover adapted to seal the tissue interface for maintaining a negative pressure at the tissue site. Such system may also comprise a canister having a collection chamber, a first or fluid inlet fluidly coupled to the collection chamber, a second or ambient inlet fluidly coupled to ambient air outside the collection chamber, a first outlet fluidly coupled to the collection chamber and adapted to receive negative pressure from a source of negative pressure, and a second outlet. Such system may further comprise a first fluid conductor fluidly coupled between the first inlet and the tissue interface, wherein the first fluid conductor may be adapted to provide negative pressure to the tissue site. Such system also may comprise a second fluid conductor fluidly coupled between the second outlet and the tissue interface, wherein the second fluid conductor may be adapted to deliver ambient air to the tissue site. In some example embodiments, the first fluid conductor and the second fluid conductor may be separate flow channels of a single conductor

In some embodiments, such system also may comprise an internal fluid conductor fluidly coupled between the second inlet and the second outlet. Such system also may comprise a regulator fluidly coupled to the second inlet, wherein the regulator is adapted to provide ambient air through the internal fluid conductor and the second fluid conductor to the tissue site. In some example embodiments, the regulator may be a filter having a known flow rate in order to deliver airflow to the tissue site in a controlled fashion. In other embodiments, for example, the regulator may be a valve adapted to vary the flow rate of ambient air to the tissue site. In some example embodiments, the regulator may be disposed within the collection chamber of the canister or outside the collection chamber between the second outlet and the second fluid conductor depending on the design of the system. In some other embodiments, the regulator may be a solenoid valve adapted to control the flow rate over time depending on the desired therapy.

In some embodiments, such system also may comprise a controller and a pressure sensor electrically coupled to the controller and fluidly coupled to the collection chamber. In some other embodiments, such systems may comprise a controller and a pressure sensor electrically coupled to the controller and fluidly coupled directly to the tissue interface. In some embodiments, the first fluid conductor may comprise a first member fluidly coupled between the first inlet and the tissue interface, and a second member fluidly coupled to the first outlet and adapted to be coupled a source of negative pressure. In such systems, the systems may further comprise a controller and a pressure sensor electrically coupled to the controller and fluidly coupled to the second member. Alternatively, such systems may further comprise a controller and a pressure sensor electrically coupled to the controller, wherein the first fluid conductor further comprises a third member fluidly coupled to the tissue interface and the pressure sensor. In some embodiments, the first member and the third member may be separate flow channels of a single conductor. In other embodiments, the second fluid conductor, and the first member and the third member of the first fluid conductor may be separate flow channels of a single conductor.

A method for providing negative pressure to a sealed space in fluid communication with a tissue site is also disclosed. In one example embodiment, the method comprises applying negative pressure through a collection chamber of a canister to the sealed space. The method may further comprise delivering ambient air to the sealed space through a fluid conductor fluidly coupled to the sealed space in response to negative pressure within the sealed space, and controlling the airflow of the ambient air by with a regulator fluidly coupled to the fluid conductor. In some embodiments, controlling the air flow of ambient air may include providing ambient air at a known flow rate, and in other embodiments controlling the air flow of ambient air may include providing ambient air at a variable flow rate. In some embodiments, the method may further comprise measuring negative pressure within the sealed space to generate negative pressure measurements, and controlling the airflow of the ambient air in response to the negative pressure measurements.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure and instillation in accordance with this specification;

FIGS. 2A and 2B are schematic diagrams illustrating additional details of a distribution system that may be associated with some example embodiments of the therapy system of FIG. 1, including some embodiments of a canister and a passive regulator;

FIGS. 3A and 3B are schematic diagrams illustrating additional details of a passive regulator that may be associated with some example embodiments of the passive regulator of FIGS. 2A and 2B, including some embodiments of a passive regulator that are filters;

FIG. 4 is a schematic diagram illustrating additional details of a distribution system that may be associated with some example embodiments of the therapy system of FIG. 1, including an example embodiment of a canister and an active regulator;

FIGS. 5 and 6 are schematic diagrams illustrating additional details of a fluid conductor that may be associated with some example embodiments of the fluid conductors of FIGS. 2A, 2B and 4, including some embodiments of fluid conductors that are separate flow channels or lumens of a single fluid conductor such as, for example, a single tube; and

FIGS. 7 and 8 are schematic diagrams illustrating additional details of a fluid conductor that may be associated with some example embodiments of the fluid conductors of FIGS. 2A, 2B and 4, including some embodiments of fluid conductors that are separate flow channels of a single fluid conductor such as, for example, a flat fluid conductor.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The term “tissue site” in this context broadly refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

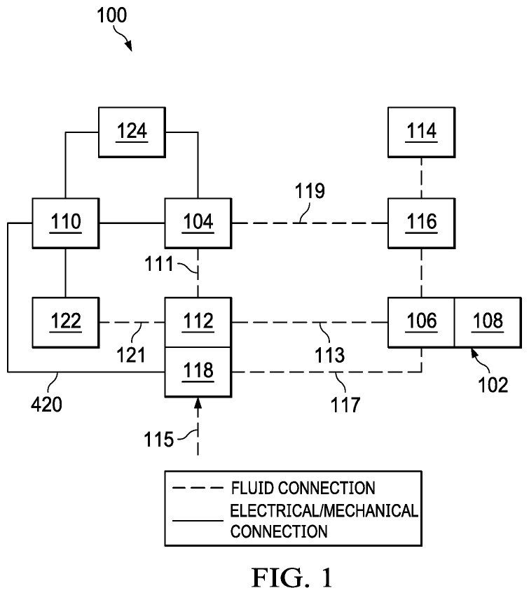

FIG. 1 is a simplified functional block diagram of an example embodiment of a therapy system 100 that can provide negative-pressure therapy with instillation of topical treatment solutions to a tissue site in accordance with this specification. The therapy system 100 may include a negative-pressure supply, and may include or be configured to be coupled to a distribution component, such as a dressing. In general, a distribution component may refer to any complementary or ancillary component configured to be fluidly coupled to a negative-pressure supply between a negative-pressure supply and a tissue site. A distribution component is preferably detachable, and may be disposable, reusable, or recyclable. For example, a dressing 102 is illustrative of a distribution component fluidly coupled to a negative-pressure source 104 in FIG. 1. A dressing may include a cover, a tissue interface, or both in some embodiments. The dressing 102, for example, may include a cover 106 and a tissue interface 108. A controller, such as a controller 110, may also be coupled to the negative-pressure source 104.

In some embodiments, another distribution component, such as a dressing interface, may facilitate coupling the negative-pressure source 104 to the dressing 102. For example, a dressing interface may be a T.R.A.C.® Pad or Sensa T.R.A.C.® Pad available from KCl of San Antonio, Tex. The therapy system 100 may optionally include a fluid container, such as a container 112, coupled to the dressing 102 and to the negative-pressure source 104 such as, for example, by fluid conductors 113 and 111, respectively.

The therapy system 100 may also include a source of instillation solution, such as a solution source 114. A distribution component may be fluidly coupled to a fluid path between a solution source and a tissue site in some embodiments. For example, an instillation pump 116 may be coupled to the solution source 114, as illustrated in the example embodiment of FIG. 1. The instillation pump 116 may also be fluidly coupled to the negative-pressure source 104 such as, for example, by a fluid conductor 119. In some embodiments, the instillation pump 116 may be directly coupled to the negative-pressure source 104, as illustrated in FIG. 1, but may be indirectly coupled to the negative-pressure source 104 through other distribution components in some embodiments. For example, in some embodiments, the instillation pump 116 may be fluidly coupled to the negative-pressure source 104 through the dressing 102

Additionally, the therapy system 100 may include sensors to measure operating parameters and provide feedback signals to the controller 110 indicative of the operating parameters. As illustrated in FIG. 1, for example, the therapy system 100 may include a pressure sensor 122, an electric sensor 124, or both, coupled to the controller 110. The pressure sensor 122 may be fluidly coupled or configured to be fluidly coupled to a distribution component such as, for example, the dressing 102 either directly or indirectly through the canister 112. The pressure sensor 122 also may be fluidly coupled to the negative-pressure source 104 indirectly through the canister 112.

Components may be fluidly coupled to each other to provide a distribution system for transferring fluids (i.e., liquid and/or gas). For example, a distribution system may include various combinations of fluid conductors and fittings to facilitate fluid coupling. A fluid conductor generally includes any structure with one or more lumina adapted to convey a fluid between two ends, such as a tube, pipe, hose, or conduit. Typically, a fluid conductor is an elongated, cylindrical structure with some flexibility, but the geometry and rigidity may vary. Some fluid conductors may be molded into or otherwise integrally combined with other components. A fitting can be used to mechanically and fluidly couple components to each other. For example, a fitting may comprise a projection and an aperture. The projection may be configured to be inserted into a fluid conductor so that the aperture aligns with a lumen of the fluid conductor. A valve is a type of fitting that can be used to control fluid flow. For example, a check valve can be used to substantially prevent return flow. A port is another example of a fitting. A port may also have a projection, which may be threaded, flared, tapered, barbed, or otherwise configured to provide a fluid seal when coupled to a component.

In some embodiments, components may also be coupled by virtue of physical proximity, being integral to a single structure, or being formed from the same piece of material. Coupling may also include mechanical, thermal, electrical, or chemical coupling (such as a chemical bond) in some contexts. For example, a tube may mechanically and fluidly couple the dressing 102 to the container 112 in some embodiments. In general, components of the therapy system 100 may be coupled directly or indirectly. For example, the negative-pressure source 104 may be directly coupled to the controller 110, and may be indirectly coupled to the dressing 102 through the container 112 by the fluid conductors 111 and 113.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

“Negative pressure” generally refers to a pressure less than a local ambient pressure, such as the ambient pressure in a local environment external to a sealed therapeutic environment provided by the dressing 102. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −75 mm Hg (−9.9 kPa) and −300 mm Hg (−39.9 kPa).

A negative-pressure supply, such as the negative-pressure source 104, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. For example, in some embodiments, the negative-pressure source 104 may be combined with the controller 110 and other components into a therapy unit. A negative-pressure supply may also have one or more supply ports configured to facilitate coupling and de-coupling the negative-pressure supply to one or more distribution components.

The tissue interface 108 can be generally adapted to contact a tissue site. The tissue interface 108 may be partially or fully in contact with the tissue site. If the tissue site is a wound, for example, the tissue interface 108 may partially or completely fill the wound, or may be placed over the wound. The tissue interface 108 may take many forms, and may have many sizes, shapes, or thicknesses depending on a variety of factors, such as the type of treatment being implemented or the nature and size of a tissue site. For example, the size and shape of the tissue interface 108 may be adapted to the contours of deep and irregular shaped tissue sites. Moreover, any or all of the surfaces of the tissue interface 108 may have projections or an uneven, course, or jagged profile that can induce strains and stresses on a tissue site, which can promote granulation at the tissue site.

In some embodiments, the tissue interface 108 may be a manifold. A “manifold” in this context generally includes any substance or structure providing a plurality of pathways adapted to collect or distribute fluid across a tissue site under pressure. For example, a manifold may be adapted to receive negative pressure from a source and distribute negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be interconnected to improve distribution or collection of fluids across a tissue site. In some illustrative embodiments, a manifold may be a porous foam material having interconnected cells or pores. For example, cellular foam, open-cell foam, reticulated foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The average pore size of a foam may vary according to needs of a prescribed therapy. For example, in some embodiments, the tissue interface 108 may be a foam having pore sizes in a range of 400-600 microns. The tensile strength of the tissue interface 108 may also vary according to needs of a prescribed therapy. For example, the tensile strength of a foam may be increased for instillation of topical treatment solutions. In one non-limiting example, the tissue interface 108 may be an open-cell, reticulated polyurethane foam such as GranuFoam® dressing or VeraFlo® foam, both available from Kinetic Concepts, Inc. of San Antonio, Tex.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic foam is a polyvinyl alcohol, open-cell foam such as V.A.C. WhiteFoam dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

The tissue interface 108 may further promote granulation at a tissue site when pressure within the sealed therapeutic environment is reduced. For example, any or all of the surfaces of the tissue interface 108 may have an uneven, coarse, or jagged profile that can induce microstrains and stresses at a tissue site if negative pressure is applied through the tissue interface 108.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 106 may provide a bacterial barrier and protection from physical trauma. The cover 106 may also be constructed from a material that can reduce evaporative losses and provide a fluid seal between two components or two environments, such as between a therapeutic environment and a local external environment. The cover 106 may be, for example, an elastomeric film or membrane that can provide a seal adequate to maintain a negative pressure at a tissue site for a given negative-pressure source. The cover 106 may have a high moisture-vapor transmission rate (MVTR) in some applications. For example, the MVTR may be at least 300 g/m\hat{ }2 per twenty-four hours in some embodiments. In some example embodiments, the cover 106 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained.

An attachment device may be used to attach the cover 106 to an attachment surface, such as undamaged epidermis, a gasket, or another cover. The attachment device may take many forms. For example, an attachment device may be a medically-acceptable, pressure-sensitive adhesive that extends about a periphery, a portion, or an entire sealing member. In some embodiments, for example, some or all of the cover 106 may be coated with an acrylic adhesive having a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

A controller, such as the controller 110, may be a microprocessor or computer programmed to operate one or more components of the therapy system 100, such as the negative-pressure source 104. In some embodiments, for example, the controller 110 may be a microcontroller, which generally comprises an integrated circuit containing a processor core and a memory programmed to directly or indirectly control one or more operating parameters of the therapy system 100. Operating parameters may include the power applied to the negative-pressure source 104, the pressure generated by the negative-pressure source 104, or the pressure distributed to the tissue interface 108, for example. The controller 110 is also preferably configured to receive one or more input signals, such as a feedback signal, and programmed to modify one or more operating parameters based on the input signals.

Sensors, such as the pressure sensor 122 or the electric sensor 124, are generally known in the art as any apparatus operable to detect or measure a physical phenomenon or property, and generally provide a signal indicative of the phenomenon or property that is detected or measured. For example, the pressure sensor 122 and the electric sensor 124 may be configured to measure one or more operating parameters of the therapy system 100. In some embodiments, the pressure sensor 122 may be a transducer configured to measure pressure in a pneumatic pathway and convert the measurement to a signal indicative of the pressure measured. In some embodiments, for example, the pressure sensor 122 may be a piezoresistive strain gauge. The electric sensor 124 may optionally measure operating parameters of the negative-pressure source 104, such as the voltage or current, in some embodiments. Preferably, the signals from the pressure sensor 122 and the electric sensor 124 are suitable as an input signal to the controller 110, but some signal conditioning may be appropriate in some embodiments. For example, the signal may need to be filtered or amplified before it can be processed by the controller 110. Typically, the signal is an electrical signal, but may be represented in other forms, such as an optical signal.

The solution source 114 is representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

The container 112 may also be representative of a container, canister, pouch, or other storage component, which can be used to collect and manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

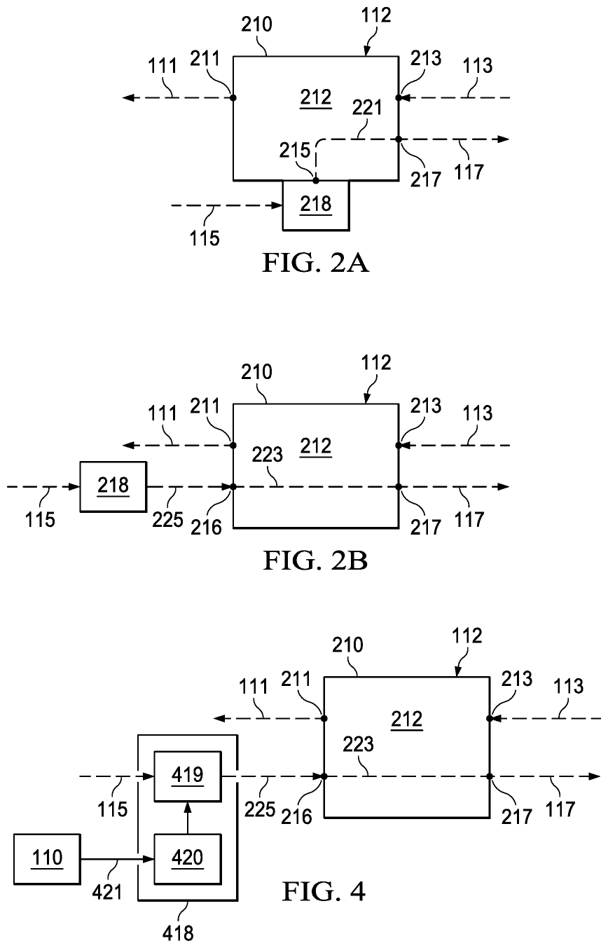

In some embodiments, the container 112 may comprise a canister having a collection chamber, a first inlet fluidly coupled to the collection chamber and a first outlet fluidly coupled to the collection chamber and adapted to receive negative pressure from a source of negative pressure. In some embodiments, a first fluid conductor may comprise a first member such as, for example, the fluid conduit 113 fluidly coupled between the first inlet and the tissue interface 108, and a second member such as, for example, the fluid conduit 111 fluidly coupled between the first outlet and a source of negative pressure whereby the first conductor is adapted to provide negative pressure within the collection chamber to the tissue site. FIGS. 2A and 2B are schematic diagrams illustrating additional details of a distribution system that may be associated with some example embodiments of the therapy system of FIG. 1, including various embodiments of the canister 112 and the regulator 118. Referring more specifically to FIG. 1 and FIGS. 2A and 2B, the container 112 in some embodiments may comprise a canister 210 having a collection chamber 212. The canister 210 may have a first inlet 213 fluidly coupled to the collection chamber 212 and the dressing 102 by the fluid conductor 113, and a first outlet 211 fluidly coupled to the collection chamber 212 and the negative-pressure source 104 by the fluid conductor 111. In some embodiments, the fluid conductors 111 and 113 may be a first fluid conductor adapted to provide negative pressure within the collection chamber 212 and to the dressing 102 and ultimately the tissue site.

The therapy system 100 may also comprise a flow regulator 218 that may be substantially similar to the regulator 118 described above. For example, the regulator 218 may be fluidly coupled by a fluid conductor to a source of ambient air outside of the container 112 to provide a controlled or managed flow of ambient air to the sealed therapeutic environment of the dressing 102 and ultimately to the tissue site. In some embodiments, the canister 210 may have a second outlet 217 fluidly coupled to the dressing 102 by the fluid conductor 117, and an ambient inlet or second inlet 215 fluidly coupled to the second outlet 217 by an internal fluid conductor 221 as shown in FIG. 2A. Although the internal fluid conductor 221 fluidly couples the second inlet 215 to the second outlet 217, the internal fluid conductor 221 is not in fluid communication with the collection chamber 212. The regulator 218 is fluidly coupled to the second inlet 215 to provide a source of ambient air as indicated by the dashed arrow 115. The regulator 218 may be a component of the container 112 having an integrated ambient inlet and outlet fluidly coupling the regulator 218 through the canister 210 to the tissue interface 108 in order to provide a source of ambient air to the tissue interface 108. For example, the regulator 218 may be located within the second inlet 215 or the internal fluid conductor 221. In other examples, the regulator 218 may form a portion of a wall of the canister 210, wherein the regulator 218 is the ambient inlet or second inlet 215.

In still other embodiments, the regulator 218 may be a separate component of the therapy system 100 that is not part of, or integrated with, the canister 210 as shown in FIG. 2B. In some embodiments, the canister 210 may have a second outlet 217 fluidly coupled to the dressing 102 by the fluid conductor 117, and an ambient inlet or second inlet 216 fluidly coupled to the second outlet 217 by an internal fluid conductor 223. Although the internal fluid conductor 223 fluidly couples the second inlet 216 to the second outlet 217, the internal fluid conductor 223 is not in fluid communication with the collection chamber 212. The regulator 218 is fluidly coupled to the second inlet 216 by an external fluid conduit 225 to provide a source of ambient air as indicated by the dashed arrow 115. The regulator 218 may be a component of the therapy system 100 having an integrated ambient inlet and outlet fluidly coupling the regulator 218 through the canister 210 to the tissue interface 108 in order to provide a source of ambient air to the tissue interface 108. For example, the regulator 218 may be located within the external fluid conductor 225.

In some embodiments, a second fluid conductor may be a single fluid conductor comprising the fluid conductor 117 fluidly coupling the regulator 218 to the tissue interface 108 and the internal fluid conductor 221 fluidly coupling the regulator 218 to a source of ambient air. In some embodiments, a second fluid conductor may be a single fluid conductor comprising the fluid conductor 117 fluidly coupling the regulator 218 to the tissue interface 108 and the internal fluid conductor 223 fluidly coupling the regulator 218 to a source of ambient air through the external fluid conductor 225. In some embodiments, the regulator 218 may be a component of the therapy system 100 or the canister 210, or proximate to the canister 210, rather than being proximate to the dressing 102 so that the air flowing through the regulator 218 is less susceptible to accidental blockage during use. In such embodiments, the regulator 218 may be attached to or positioned proximate the canister 210 and/or proximate a source of ambient air where the regulator 218 is less likely to be blocked during use if the regulator were disposed proximate the dressing 102.

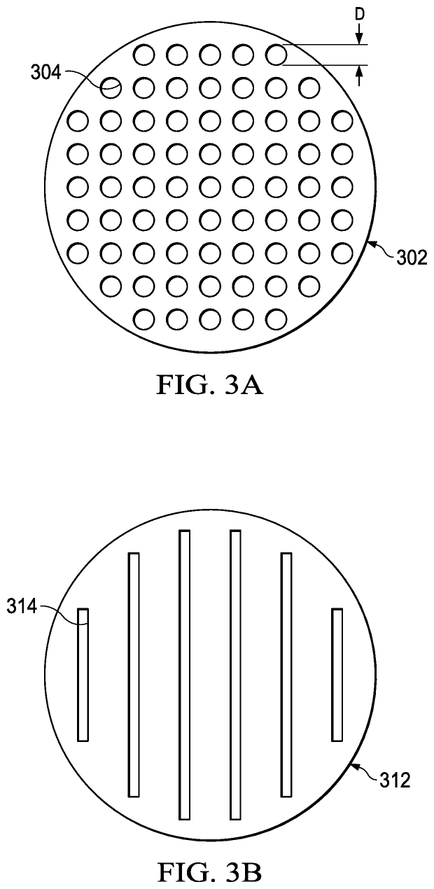

A regulator may be any device for controlling fluid flow and, more specifically, for controlling air flow. Airflow regulators may include constant airflow regulators such as, for example, the regulator 218 shown in FIGS. 2A and 2B that may also be referred to as passive regulators. In some embodiments, a passive regulator may be a device having a single opening with a known flow rate or a filter having a plurality of openings with a known flow rate, or variable regulators such as, for example, a solenoid valve or a needle valve. In some embodiments, the airflow regulator may comprise a filter that may be a hydrophillic/oeliophillic, bacterial filter having a known flow rate. Referring to FIGS. 3A and 3B, alternative example embodiments of the regulator 218 are shown that may include, for example, filters 302 and 312, respectively. In one embodiment, the filter 302 may comprise a plurality of micro-pores 304 that may have a diameter (D) in a range between 0.25 and 1.0 μm. The quantity of micro-pores 304 may be varied to so that the cumulative flow rate through the micro-pores 304 is sufficient for the flow of ambient air into the tissue interface 108. The micro-pores 304 may be formed in a pattern or in a random fashion. The micro-pores 304 may be further sized to function as a barrier to bacteria or viruses to mitigate the possibility of infection at the tissue site. In yet another embodiment, the filter 312 may be formed from a plurality of micro-slits 314 that are sized with a lateral gap such that the micro-slits 314 function analogously to the micro-pores 304. Other embodiments of filters may include a grating consisting of perpendicular micro-slits (not shown) similar to those described above with reference to the micro-slits 314.

In some embodiments, the filters 302 and 312 described above function as passive devices because they only control the flow of air during intervals of negative pressure when a negative pressure is present at, or being applied to, the tissue interface 108 that draws ambient air through the regulator 218 at the predetermined flow rate. Alternatively, the regulator 118 may comprise an active device such as, for example, a solenoid valve that can be controlled during intervals of negative pressure. Referring to FIG. 4 for example, the regulator 118 may be a regulator 418 comprising a solenoid valve 419 coupled to an actuating device 420 that is electrically coupled to the controller 110 by an electrical conductor 421. In some embodiments, the controller 110 may be programmed to actuate the solenoid valve 419 to release ambient air to the tissue interface 108 for predetermined flow-control intervals of time during the negative pressure intervals. In yet other embodiments, the controller 110 may be programmed to control the solenoid valve 419 to vary the flow rate during the predetermined flow-control intervals. The solenoid valve 419 also may be a component of the therapy system 100 or the canister 210 as described above with respect to the positioning of the regulator 218.

In operation, the tissue interface 108 may be placed within, over, on, or otherwise proximate to a tissue site. The cover 106 may be placed over the tissue interface 108 and sealed to an attachment surface near the tissue site. For example, the cover 106 may be sealed to undamaged epidermis peripheral to a tissue site. Thus, the dressing 102 can provide a sealed therapeutic environment proximate to a tissue site, substantially isolated from the external environment, and the negative-pressure source 104 can reduce the pressure in the sealed therapeutic environment. Negative pressure applied across the tissue site through the tissue interface 108 in the sealed therapeutic environment can induce macrostrain and microstrain in the tissue site, as well as remove exudates and other fluids from the tissue site, which can be collected in container 112. In some embodiments, negative pressure may be applied intermittently or periodically, with intervals of negative pressure being applied to the tissue interface 108 and drawing ambient air through the regulator 118. In some embodiments, ambient air may be drawn through the regulator 118 at a predetermined flow rate using, for example, the regulator 218 as described above. In other embodiments, ambient air may be drawn through the regulator 118 at a variable flow rate using, for example, the regulator 418 as described above.

The therapy system 100 may also comprise a sensing device such as, for example, a flow sensor (not shown) that directly measures the flow rate of air between the regulator 118 (including the regulator 218 and the regulator 418) and tissue interface 108, i.e., the flow rate (FR). The flow sensor may be, for example, a flow-meter or a differential processor for computing the time rate of change in the difference between the pressure at the tissue site, i.e., the wound pressure (WP), and the pressure at the negative pressure source 104, i.e., the pump pressure (PP). The flow rate (FR) may be measured, for example, in units of cubic centimeters of air per minute (cc/min), between the negative pressure source 104 and the tissue site. The flow rate (FR) provides some indication of the extent to which the dressing 102 or other components of the negative pressure system 100 might be leaking to reduce the pressure at the tissue site below the desired pressure targeted for therapy. For example, a high flow rate (FR) might indicate that the dressing 102 or other components of the therapy system 100 are considered to be in a “high leakage condition” requiring the pump of the negative pressure source 104 to continue running or run more frequently in order to offset the higher leakage. Alternatively, a lower flow rate (FR) might indicate that the dressing 115 or other components of the system 100 are considered to be in a more efficient “low leakage condition” requiring the pump of the negative pressure source 104 to run intermittently or less frequently. In some embodiments, the dressing 102 and other components in the system might be considered to have a fairly high leakage rate (LR) of approximately 300 cc/min and a fairly low leakage rate (LR) of approximately 50 cc/min.

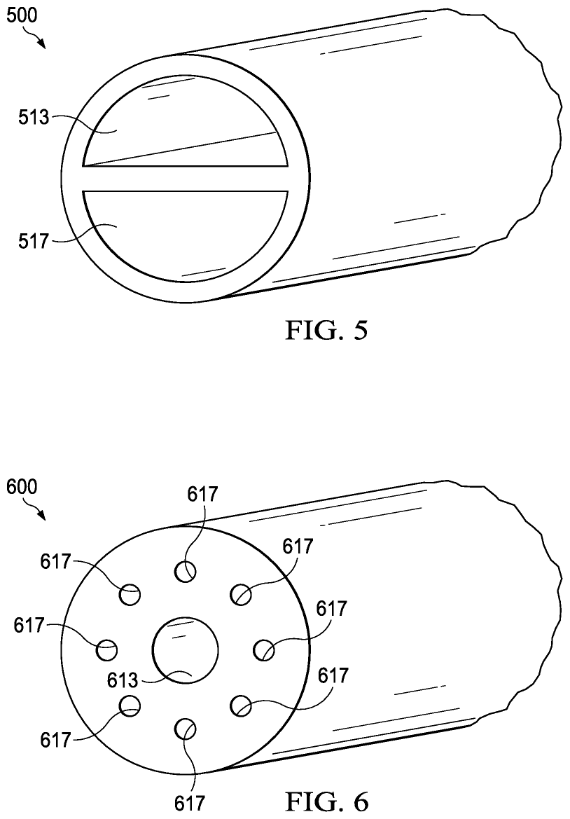

As indicated above, the fluid conductors 113 and 117 may be separate lumens disposed in a single fluid conductor. Referring to FIG. 5, for example, the fluid conductors 113 and 117 may be implemented in a single multi-lumen tube 500 comprising two lumens including lumens 513 and 517 that may correspond to the fluid conductors 113 and 117, respectively. When negative pressure is applied, exudates and other fluids are drawn from the tissue site into the canister 210 through the lumen 513, and ambient air is drawn into the tissue interface 108 from the regulator 218 through the lumen 517. In yet another embodiment shown in FIG. 6, the fluid conductors 113 and 117 may be disposed in a single multi-lumen tube 600 comprising a central lumen 613 and a plurality of peripheral lumens 617 corresponding to the fluid conductors 113 and 117, respectively. When negative pressure is applied, exudates and other fluids are drawn from the tissue site into the canister 210 through the central lumen 613 which may have a larger diameter than the peripheral lumens 617 to accommodate the volume of exudates and other fluids, and ambient air is drawn into the tissue interface 108 from the regulator 218 through the peripheral lumens 617. In another embodiment wherein the pressure sensor 122 is fluidly coupled directly to the dressing 102 by a third fluid conductor (not shown), the third fluid conductor may also be one or more of the peripheral lumens 617 disposed in the tube 600.

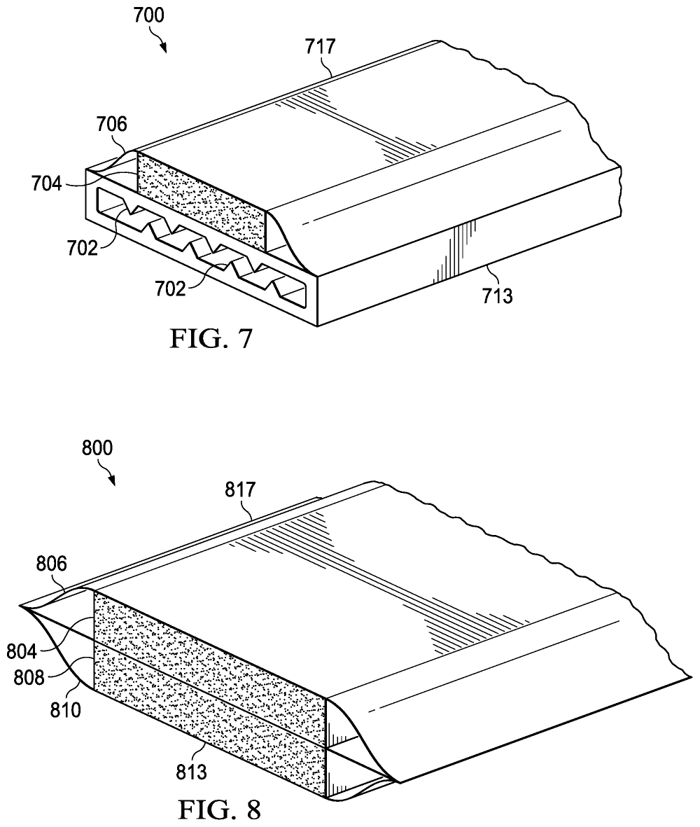

The fluid conductors 113 and 117 may be separate lumens in a single fluid conductor having a variety of different shapes other than a tube such as multi-lumen tubes 500 and 600. Referring to FIG. 7, for example, the fluid conductors 113 and 117 may be implemented in a single multi-lumen conduit 700 that may be substantially flat and comprise two lumens including lumens 713 and 717 that may correspond to the fluid conductors 113 and 117, respectively. The lumen 713 may comprise a semi-rigid material and have a cross-section that may be generally rectangular in shape with an opening that is generally rectangular in shape. The lumen 713 may further comprise spacers disposed inside of the opening to prevent the lumen 713 from collapsing when subjected to compressive loads that are caused either by internal negative pressure or an external weight. In some embodiments for example, the spacers may be protrusions 702 extending inwardly from one side of the opening toward the other side of the opening of the lumen 713. Such protrusions may have a variety of shapes including triangular or cylindrical shapes that are arranged in a pattern or in a random fashion within the lumen 713.

The lumen 717 may comprise a foam material 704 covered by a drape 706 coupled to the lumen 713. In some embodiments, the foam material 704 may comprise a an open-cell, reticulated polyurethane foam such as GranuFoam® dressing or VeraFlo® foam, both available from Kinetic Concepts, Inc. of San Antonio, Tex. In some embodiments, the drape 706 may be an elastomeric film or membrane that can provide a seal adequate to maintain a negative pressure within the lumen 717. When negative pressure is applied, exudates and other fluids are drawn from the tissue site into the canister 210 through the lumen 713, and ambient air is drawn into the tissue interface 108 from the regulator 218 through the lumen 717.

Referring to FIG. 8, for example, the fluid conductors 113 and 117 may be implemented in a single multi-lumen conduit 800 that also may be substantially flat and comprise two lumens including lumens 813 and 817 that may correspond to the fluid conductors 113 and 117, respectively. The lumen 817 may comprise a foam material 804 encapsulated by a cover 806 coupled to the lumen 813. In some embodiments, the foam material 804 may comprise a an open-cell, reticulated polyurethane foam such as GranuFoam® dressing or VeraFlo® foam, both available from Kinetic Concepts, Inc. of San Antonio, Tex. In some embodiments, the cover 806 may be an elastomeric film or membrane that can provide a seal adequate to maintain a negative pressure within the lumen 817.

The lumen 813 may comprise a spacer or a filler material 808 encapsulated by a cover 810 coupled to the lumen 817. In some embodiments, the cover 806 may be an elastomeric film or membrane that can provide a seal adequate to maintain a negative pressure within the lumen 813. The filler material 808 may also comprise foams or non-woven materials as long as they are sufficiently rigid for preventing the lumen 813 from collapsing when subjected to compressive loads that are caused either by internal negative pressure or an external weight. In some embodiments, the filler material 808 may comprise 3D materials such as, for example, 3D textiles, foams of higher stiffness, or extruded polymer foams similar to those used for fluid drains. When negative pressure is applied, exudates and other fluids are drawn from the tissue site into the canister 210 through the lumen 813, and ambient air is drawn into the tissue interface 108 from the regulator 218 through the lumen 817.

The systems, apparatuses, and methods described herein may provide significant advantages. For example, some therapy systems include fluid conductors arranged in a closed system that does not provide airflow to a tissue interface frequently enough may result in the creation of a significant head pressure. The head pressure in some embodiments may be defined as a difference in pressure (DP) between a negative pressure set by a user or caregiver for treatment, i.e., the target pressure (TP), and the negative pressure provided by a negative pressure source that is necessary to offset the pressure drop inherent in the fluid conductors, i.e., the supply pressure (SP), in order to achieve or reach the target pressure (TP). For example, the head pressure that a negative pressure source needs to overcome may be as much as 75 mmHg. Problems may occur in such closed systems when a blockage occurs in the pneumatic pathway of the fluid conductors that causes the negative pressure source to increase to a value above the normal supply pressure (SP) as a result of the blockage. For example, if the blockage suddenly clears, the instantaneous change in the pressure being supplied may cause harm to the tissue site. Consequently, the supply pressure (SP) is limited to a maximum value that cannot be exceeded in order to avoid the possibility of causing harm to the tissue site.

Some therapy systems have attempted to compensate for head pressure by introducing a supply of ambient air flow into the therapeutic environment and the pneumatic pathway of the fluid conductors by providing a vent on the dressing to provide ambient air flow into the therapeutic environment at a controlled leak. Such a vent may also utilize a filter that could become blocked when the dressing is applied or if the user or patient accidentally sits on the vent after the dressing is applied. Locating the filter in such a location may also be problematic because it is more likely to be contaminated or compromised by other chemicals and agents associated with treatment utilizing instillation fluids that could adversely affect the performance of the filter and the vent itself.

The embodiments of the therapy systems described above clearly overcome the problems associated with having a large head pressure in a closed pneumatic environment, and the problems associated with using a vent disposed on or adjacent the dressing intending to provide airflow or a controlled leak to the therapeutic environment. More specifically, the embodiments of the therapy systems described above include a fluid conductor fluidly coupled to the therapeutic environment and to a fluid regulator co-located proximate to the canister or the housing of the therapy system, but separated from the dressing itself. In embodiments of therapy systems that include an air flow regulator comprising a filter and a fluid conductor as described above, the filter maintains a substantially constant airflow and provides a continuous flow of a mixture of wound fluids and ambient air into the canister as described above. Moreover, such embodiments reduce the head pressure associated with the fluid conductors of the therapeutic system so that the negative pressure source can achieve the same target pressure (TP) with a lower supply pressure (SP). Such therapy systems utilizing an air flow regulator as described above are not only safer, but also require less battery power to generate the same target pressure (TP). Such therapeutic systems including airflow regulators also facilitate detection of blockages in the fluid conductors because erroneous blockages will be less likely to be confused with the elimination of a systemic leak.

In embodiments of therapy systems that include an air flow regulator comprising a valve such as the solenoid valve described above, the valve provides a controlled airflow as opposed to a constant airflow. The valve of the air flow regulator otherwise possesses many similarities to the filter embodiment and the same benefits as described above. The controller may be programmed to periodically open the solenoid valve as described above allowing ambient air to flow into the fluid conduit and tissue interface for a predetermined duration of time and consequently providing a predetermined volume of airflow into the pneumatic system of fluid conductors. This feature allows the controller to activate the solenoid valve in a predetermined fashion to purge any blockages that may develop in the fluid conductors during operation. In some embodiments, the controller may be programmed to open the solenoid valve for a fixed period of time at predetermined intervals such as, for example, for five seconds every four minutes to mitigate the formation of any blockages.

In some other embodiments, the controller may be programmed to open the solenoid valve in response to a stimulus within the pneumatic system of fluid conductors rather than, or additionally, being programmed to function on a predetermined therapy schedule. For example, if the pressure sensor is not detecting pressure decay in the canister, this may be indicative of a column of fluid forming in the fluid conduits or the presence of a blockage in the fluid conduits. Likewise, the controller may be programmed to recognize that an expected drop in canister pressure as a result of the valve opening may be an indication that the fluid conductors are open. The controller may be programmed to conduct such tests automatically and routinely during therapy so that the patient or caregiver can be forewarned of an impending blockage. The controller may also be programmed to detect a relation between the extent of the deviation in canister pressure resulting from the opening of the valve and the volume of fluid with in the fluid conductors. For example, if the pressure change within the canister is significant when measured, this could be an indication that there is a significant volume of fluid within the fluid conductors. However, if the pressure change within the canister is not significant, this could be an indication that the plenum volume was larger.

In some other embodiments, the controller may be programmed to infer information about the fluid in a pneumatic system from the delay between the opening of the solenoid valve and the measured change in the canister pressure. For example, the quantity, viscosity and thickness of the exudate in the first fluid conduit between the dressing 102 and the canister 210 may be inferred from the delay in the pressure change within the canister. If the first fluid conductor is substantially open, the change in pressure within the canister would be faster than when the first fluid conductor is more occluded. Consequently, if the first fluid conductor is occluded as indicated by a more sluggish change in pressure within the canister, the controller may be programmed to activate the valve more frequently for longer periods of time until the change in pressure increases to an acceptable level.

A method for providing negative pressure to a sealed space in fluid communication with a tissue site is also disclosed. In one example embodiment, the method comprises applying negative pressure through a collection chamber of a canister to the sealed space. The method may further comprise delivering ambient air to the sealed space through a fluid conductor fluidly coupled to the sealed space in response to negative pressure within the sealed space, and controlling the airflow of the ambient air by with a regulator fluidly coupled to the fluid conductor. In some embodiments, controlling the air flow of ambient air may include providing ambient air at a known flow rate, and in other embodiments controlling the air flow of ambient air may include providing ambient air at a variable flow rate. In some embodiments, the method may further comprise measuring negative pressure within the sealed space to generate negative pressure measurements, and controlling the airflow of the ambient air in response to the negative pressure measurements.

The systems, apparatuses, and methods described herein may provide other significant advantages. For example, when the first and second fluid conductors are combined into a single fluid conductor as described above, the single fluid conductor may simplify use of the system. Additionally, the single fluid conductor may be fluidly coupled directly to the canister allowing the user or caregiver to connect only one conductor to the therapy system rather than two separate fluid conductors.

The disposable elements can be combined with the mechanical elements in a variety of different ways to provide therapy. For example, in some embodiments, the disposable and mechanical systems can be combined inline, externally mounted, or internally mounted.

While shown in a few illustrative embodiments, a person having ordinary skill in the art will recognize that the systems, apparatuses, and methods described herein are susceptible to various changes and modifications. For example, certain features, elements, or aspects described in the context of one example embodiment may be omitted, substituted, or combined with features, elements, and aspects of other example embodiments. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 102, the container 112, or both may be eliminated or separated from other components for manufacture or sale. In other example configurations, the controller 110 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

What is claimed is:

1. A system for providing negative pressure to a tissue site, the system comprising:
a dressing having tissue interface in fluid communication with the tissue site and a cover adapted to seal the tissue interface for maintaining a negative pressure at the tissue site;
a canister having a collection chamber, a first inlet fluidly coupled to the collection chamber, a second inlet fluidly coupled to ambient air outside the collection chamber, a first outlet fluidly coupled to the collection chamber and adapted to receive negative pressure from a source of negative pressure, and a second outlet;
a first fluid conductor fluidly coupled between the first inlet and the tissue interface, the first fluid conductor adapted to provide negative pressure to the tissue site;
a second fluid conductor fluidly coupled between the second outlet and the tissue interface, the second fluid conductor adapted to deliver ambient air to the tissue site;
an internal fluid conductor fluidly coupled between the second inlet and the second outlet; and
a regulator fluidly coupled to the second inlet, the regulator adapted to provide ambient air to the second fluid conductor through the internal fluid conductor.

2. The system of claim 1, wherein the regulator is coupled to the second inlet of the canister.

3. The system of claim 1, wherein the regulator is disposed on a surface of the canister.

4. The system of claim 1, further comprising an external fluid conductor fluidly coupled between the regulator and the second inlet, wherein the regulator is disposed within a housing of the system separate from the dressing and the canister.

5. The system of claim 1, wherein the regulator is a filter.

6. The system of claim 5, wherein the filter is a bacterial filter.

7. The system of claim 5, wherein the filter is a hydrophilic filter.

8. The system of claim 5, wherein the filter has a known flow rate.

9. The system of claim 5, wherein the filter includes an orifice having a known flow rate.

10. The system of claim 5, wherein the filter includes a plurality of orifices having a known flow rate.

11. The system of claim 1, wherein the regulator is a valve.

12. The system of claim 11, wherein the valve has a known flow rate.

13. The system of claim 11, wherein the valve has a variable flow rate.

14. The system of claim 11, wherein the valve is a solenoid valve adapted to vary the flow rate of valve.

15. The system of claim 14, further comprising a controller electrically coupled to the solenoid valve and adapted to receive an input for varying the flow rate over time.

16. The system of claim 15, wherein the controller receives an input for providing an intermittent flow rate.

17. The system of claim 15, wherein the controller receives an input for providing a variable flow rate.

18. The system of claim 1, wherein the first fluid conductor and the second fluid conductor are separate flow channels of a single conductor.

19. The system of claim 1, further comprising a controller and a pressure sensor electrically coupled to the controller and fluidly coupled to the collection chamber.

20. The system of claim 1, further comprising a controller and a pressure sensor electrically coupled to the controller and fluidly coupled directly to the tissue interface.

21. The system of claim 1, wherein the first fluid conductor comprises a first member fluidly coupled between the first inlet and the tissue interface, and a second member fluidly coupled to the first outlet and adapted to be coupled a source of negative pressure.

22. The system of claim 21, further comprising a controller and a pressure sensor electrically coupled to the controller and fluidly coupled to the second member.

23. The system of claim 21, further comprising a controller and a pressure sensor electrically coupled to the controller, and wherein the first fluid conductor further comprises a third member fluidly coupled to the tissue interface and the pressure sensor.

24. The system of claim 23, wherein the first member and the third member are separate flow channels of a single conductor.

25. The system of claim 23, wherein the second fluid conductor, and the first member and the third member of the first fluid conductor are separate flow channels of a single conductor.

26. An apparatus for providing negative pressure to a sealed space through a dressing connector in fluid communication with a tissue site, the apparatus comprising:
a canister having a collection chamber, a fluid inlet fluidly coupled to the collection chamber, an ambient inlet, a first outlet fluidly coupled to the collection chamber and adapted to receive negative pressure from a source of negative pressure, and a second outlet;
a first tube adapted to be fluidly coupled between the fluid inlet and the dressing connector to provide negative pressure to the sealed space;
a second tube adapted to be fluidly coupled between the second outlet and the dressing connector to deliver ambient air to the sealed space;
a third tube fluidly coupled between the second outlet and an ambient input; and
a regulator fluidly coupled to the ambient inlet, the regulator adapted to provide ambient air to the second conductor through the third tube.

27. The apparatus of claim 26, wherein the regulator is a filter.

28. The apparatus of claim 26, wherein the regulator is a filter disposed proximate the ambient inlet.

29. The apparatus of claim 26, wherein the regulator is a valve.

30. The apparatus of claim 26, wherein the regulator is a valve disposed proximate the ambient inlet.

31. The apparatus of claim 26, further comprising a fourth tube fluidly coupled between the regulator and the ambient inlet, wherein the regulator is disposed within a housing of the apparatus separate from the canister.

32. The apparatus of claim 26, further comprising a flow sensor fluidly coupled between the regulator and the ambient inlet.

33. A method for providing negative pressure to a sealed space in fluid communication with a tissue site, the method comprising:
applying negative pressure through a collection chamber of a canister to the sealed space;
delivering ambient air to the sealed space through a fluid conductor fluidly coupled to the sealed space in response to negative pressure within the sealed space; and
controlling the airflow of the ambient air with a regulator fluidly coupled to the fluid conductor.

34. The method of claim 33, wherein controlling the air flow of ambient air includes providing ambient air at a known flow rate.

35. The method of claim 33, wherein controlling the air flow of ambient air includes providing ambient air at a variable flow rate.

36. The method of claim 33, wherein controlling the air flow of ambient air includes providing ambient air at an intermittent flow rate.

37. The method of claim 33, further comprising measuring negative pressure within the sealed space to generate negative pressure measurements and controlling the airflow of the ambient air in response to the negative pressure measurements.

38. The systems, apparatuses, and methods substantially as described herein.
